(19) United States
(12) Patent Application Publication
(10) Pub. No.: US 2026/0124039 A1
(43) Pub. Date: May 7, 2026

(54) Expandable Spacer with Internal Expansion Actuator

(71) Applicant: DeGen Medical, Inc., Florence, SC (US)

(72) Inventor: Charles Lucasiewicz, Florence, SC (US)

(73) Assignee: DeGen Medical, Inc., Florence, SC (US)

(21) Appl. No.: 19/382,853

(22) Filed: Nov. 7, 2025

Related U.S. Application Data
(60) Provisional application No. 63/717,485, filed on Nov. 7, 2024.

(51) Int. Cl. A61F 2/44; A61F 2/30

(52) U.S. Cl. CPC A61F 2/44; A61F 2002/30153; A61F 2002/30261; A61F 2002/30317; A61F 2002/30387; A61F 2002/30433; A61F 2002/30556; A61F 2002/30621

(57) ABSTRACT

The description relates to an expandable intervertebral spacer configured to engage an intervertebral disk. An example expandable spacer includes a main body, a first endplate, a second endplate, a driving member, a plurality of pins, and an actuation member. The expandable spacer is configured to transition from a first configuration to a second configuration by various structures (e.g., steps, faceted surfaces, curved surfaces, multi-faceted portions) defined on the first endplate, the second endplate, and the driving member.

### FIELD

The disclosure relates to the field of implantable medical devices. More particularly, the disclosure relates to medical devices suitable for implantation in spaces between bones, such as spaces between vertebral bodies in a spinal column of a vertebrate or between other bones. Specific examples relate to expandable spacers suitable for implantation between adjacent vertebral bodies in a spinal column. The disclosure also relates to methods of making expandable spacers and methods of supplying expandable spacers.

### BACKGROUND

Bone degeneration can be caused by trauma, disease, and natural processes, such as aging, which can have a negative impact on the lifestyle of an animal. For example, destabilization of a spine in a vertebrate, such as a human being, may result in alteration of the spacing between the adjacent vertebral bodies. This destabilization can place pressure onto the surrounding nerves and tissues between the vertebral bodies causing pain, discomfort, and, eventually, nerve damage.

One approach to alleviating the pain and discomfort caused by the destabilization of the spacing between the adjacent vertebral bodies is to implant a medical device commonly referred to as an intervertebral spacer, or simply a spacer, into the space between two adjacent vertebral bodies. The intervertebral spacer supports the structure of the spine by maintaining a desired spacing between adjacent intervertebral bodies and proper angular positioning of the spinal column.

Some intervertebral spacers are static devices that provide a spacer having fixed dimensions. Expandable intervertebral spacers are dynamic devices that allow for controlled expansion in situ. These expandable spacers provide several benefits, including reduction of the trialing required to identify and select an appropriate spacer for implantation and reduction of impaction of the adjacent vertebral bodies that can occur during insertion. Overall, expandable spacers offer a clinician an ability to optimize the fit of the spacer between intervertebral bodies during placement.

Despite the existence of various expandable spacers in the art, a need for improved expandable intervertebral spacers remains.

### BRIEF SUMMARY OF SELECTED EXAMPLES

An example is an expandable spacer. An expandable spacer includes a terminal body that defines a top groove and a threaded terminal aperture, which further defines a central axis. The spacer also comprises a driving member defining a driving aperture, a first top rail, and a first bottom rail. A bolt with both a headed portion and a threaded portion is disposed within the terminal body and the driving member. The top endplate features an outer surface spaced apart from the central axis and includes a top tongue disposed within the top groove. It also has a first top guide track in which the first top rail is disposed; this guide track defines a first top ramp angle with respect to the central axis. The bottom endplate includes a first bottom guide track in which the first bottom rail is disposed. When the bolt is rotated, the headed portion of the bolt drives the top endplate along the first top ramp angle, increasing the space between the top endplate outer surface and the central axis.

### DESCRIPTION OF FIGURES

FIG. 1 is a perspective view of an example expandable spacer. The expandable spacer is shown in the first configuration.

FIG. 2 is a perspective view of the example expandable spacer illustrated in FIG. 1. The expandable spacer is shown in the second configuration.

FIG. 3 is a perspective view of the driving member disposed within the expandable spacer illustrated in FIG. 1.

FIG. 4 is a perspective view of the top endplate presented in the expandable spacer illustrated in FIG. 1.

FIG. 5 is a perspective view of the bottom endplate presented in the expandable spacer illustrated in FIG. 1.

FIG. 6 is an exploded view of the example expandable spacer illustrated in FIG. 1.

FIG. 7 is a top view of the example expandable spacer illustrated in FIG. 1. The expandable spacer is shown in the first configuration.

FIG. 8 is a bottom view of the example expandable spacer illustrated in FIG. 1. The expandable spacer is shown in the first configuration.

FIG. 9 is back view of the example expandable spacer illustrated in FIG. 1. The expandable spacer is shown in the first configuration.

FIG. 10 is front view of the example expandable spacer illustrated in FIG. 1. The expandable spacer is shown in the first configuration.

### DESCRIPTION OF SELECTED EXAMPLES

The following detailed description and the appended drawings describe and illustrate various example expandable spacers, example methods of making expandable spacers, and example methods of supplying expandable spacers. The description and drawings are provided to enable one skilled in the art to make and use one or more example expandable spacers. They are not intended to limit the scope of the claims in any manner.

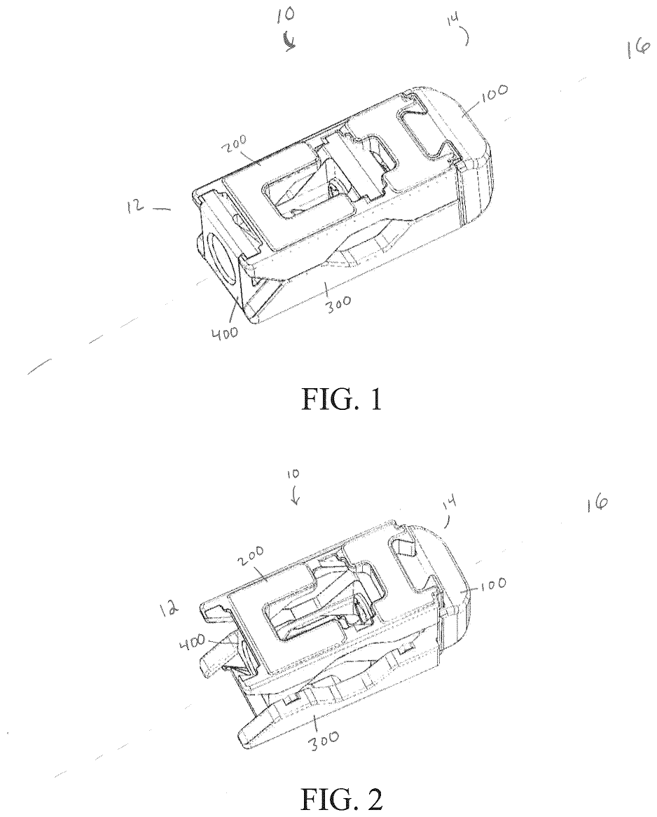

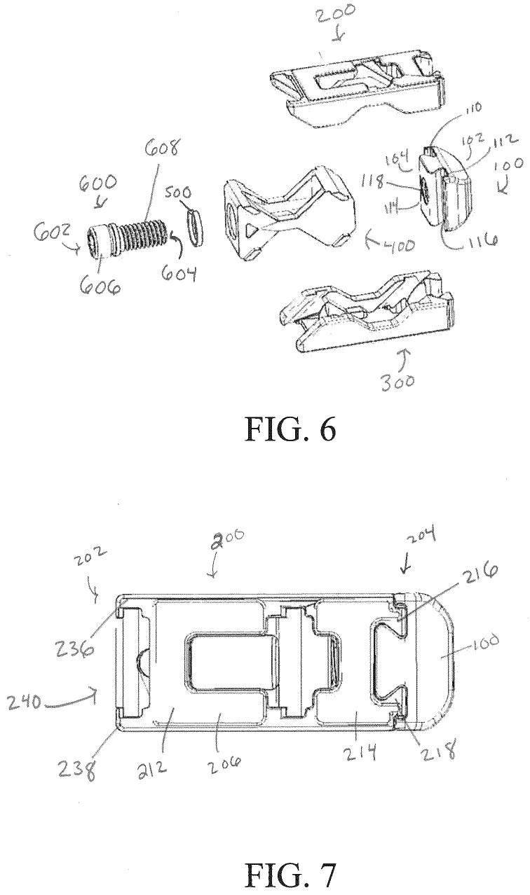

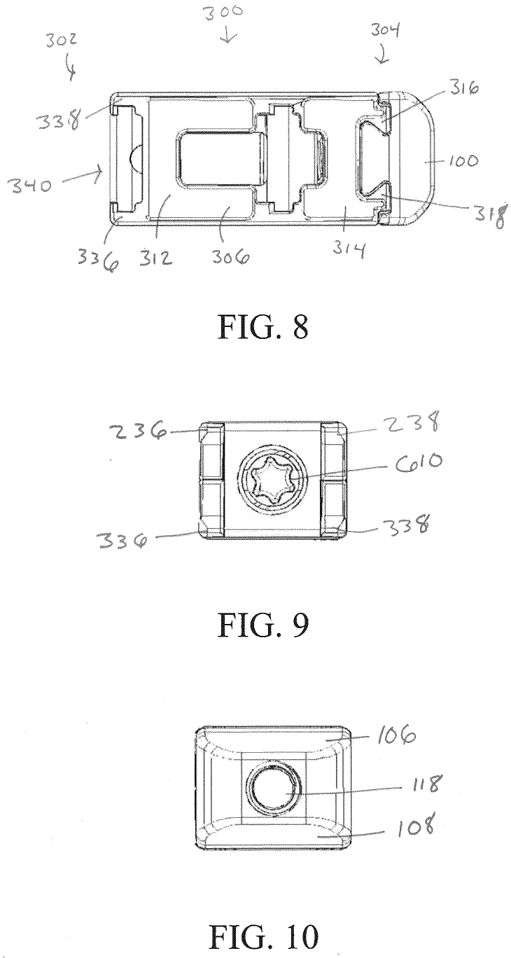

Each of FIGS. 1, 2, 3, 4, 5, 6, 7, 8, 9, and 10 illustrates an example expandable spacer 10. The expandable spacer 10 comprises a terminal body 100, a first endplate 200, a second endplate 300, a driving member 400, a retention member 500, and an actuation member 600.

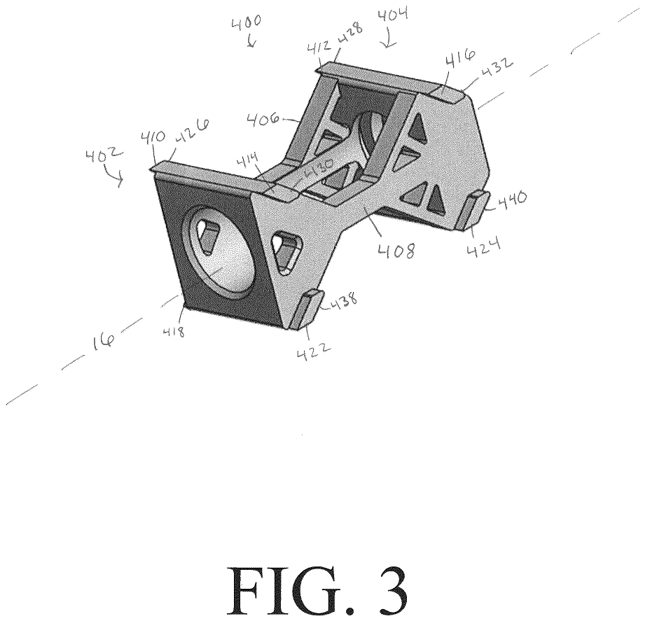

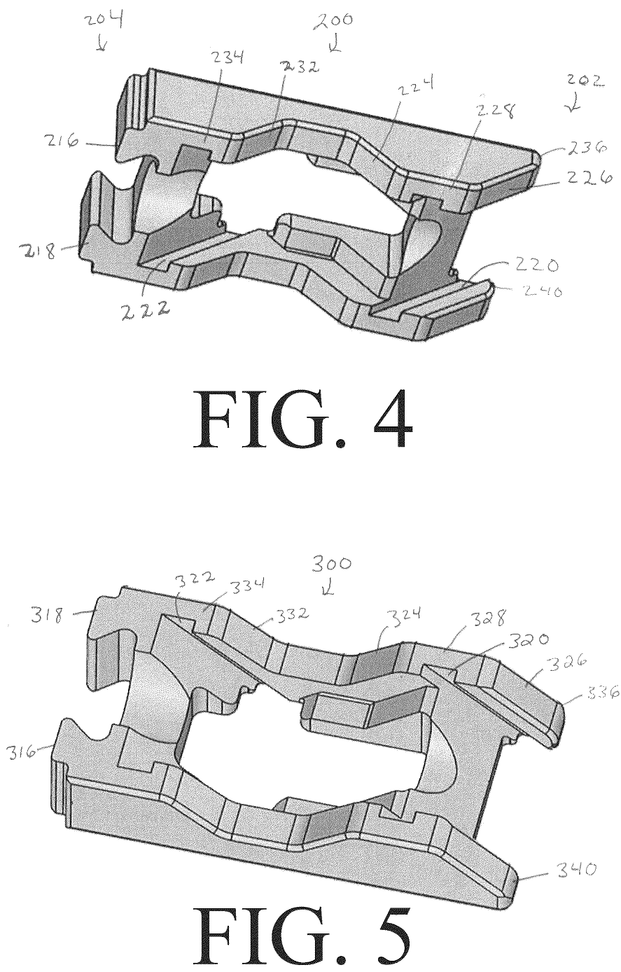

The expandable spacer 10 is movable between a first configuration and a second configuration. FIG. 1 illustrates the expandable spacer 10 in the first configuration. FIG. 2 illustrates the expandable spacer 10 in the second configuration. The expandable spacer 10 moves between the first configuration and the expanded configuration as a result of rotational movement of the actuation member 600, which increases or decreases the spacing between the first endplate 200 and the second endplate 300, as described below. Each of FIGS. 3, 4, and 5 illustrates the expandable spacer 10 in a particular expansion state and this group of figures illustrates transition of the expandable spacer 10 from the first configuration to the second configuration. FIG. 3 illustrates the expandable spacer 10 in the first configuration while FIG. 5 illustrates the expandable spacer 10 in the second configuration. FIG. 4 illustrates the expandable spacer 10 in a state of expansion that is between the first configuration and the second configuration. In use, the expandable spacer 10 can be implanted in any state of expansion that exists between the first configuration and the second configuration.

The first configuration, illustrated in FIGS. 1, 3, 7, 8, 9, and 10, can be referred to as an unexpanded configuration. In this configuration, each of the first endplate 200 and second endplate 300 interfaces with the terminal body 100. Also, the actuation member 600 is in a first position, which places the driving member 400 in a first position. The second configuration, illustrated in FIG. 2, can be referred to as an expanded configuration. In this configuration, each of the first endplate 200 and second endplate 300 is spaced such that the distance between the first and second endplates 200, 300 has increased as compared to the first configuration. Also, the actuation member 600 is in a second position, achieved by rotating the actuation member 600, which moves the driving member 400 to a second position. The expandable spacer 10 moves between the first configuration and the second configuration through rotational movement of the actuation member 600, which forces the driving member 400 to move linearly along a longitudinal axis of the expandable spacer 10 toward the terminal body 100. As a result of the structural interfaces between the driving member 400, first endplate 200, and second endplate 300, this linear movement of the driving member 400 forces the first endplate 200 and second endplate 300 away from each other in opposing directions along an axis transverse to the longitudinal axis of the expandable spacer 10.

The spacer 10 is an implantable medical device used for vertebral alignment by maintaining or restoring proper spacing between adjacent vertebral bodies in the spinal column. Extending from a spacer proximal end 12 to a spacer distal end 14 along a central axis 16, the spacer proximal end 12 is closer to the user of the device, such as a surgeon, while the spacer distal end 14 is the opposite end, positioned deeper within the vertebral space. The spacer 10 is expandable, allowing for controlled adjustment during implantation to achieve the desired vertebral alignment. The ability to expand facilitates the accommodation of patient-specific anatomical variations and aids in decompressing neural elements affected by spinal disorders. The spacer 10 includes several main components: a terminal body 100, a driving member 400, a bolt 600, a top endplate 200, and a bottom endplate 300.

The terminal body 100 connects to both the top endplate 200 and the bottom endplate 300. It serves as the main structural element, housing internal mechanisms and providing attachment points for other components. The driving member 400 is positioned within the spacer 10 and interacts with the bolt 600. It is configured to translate rotational motion from the bolt 600 into linear movement, facilitating the expansion or contraction of the spacer 10. The bolt 600 extends through the terminal body 100 and the driving member 400, connecting at the spacer proximal end 12. When rotated, the bolt 600 actuates the driving member 400, causing the spacer 10 to expand or contract as needed. The top endplate 200 is attached to the terminal body 100 at the upper side and interfaces with the superior vertebral body. It provides a surface for load distribution and maintains contact with the adjacent bone. The bottom endplate 300 is connected to the terminal body 100 at the lower side and interfaces with the inferior vertebral body. Similar to the top endplate 200, it ensures proper load distribution and bone contact.

The terminal body 100 is distally located within the spacer 10. It connects to the top endplate 200 at the terminal body top surface 106 and to the bottom endplate 300 at the terminal body bottom surface 108. The terminal body 100 acts as the main structural component of the spacer 10. The terminal body 100 includes a terminal body proximal surface 102. It also includes a distal surface 104. Additionally, the terminal body 100 includes a terminal body top surface 106, and a terminal body bottom surface 108.

The terminal body proximal surface 102 defines each of a first terminal body top groove 110, a second terminal body top groove 112, a first terminal body bottom groove 114, and a second terminal body bottom groove 116. In some embodiments, the terminal body 100 may require only a single groove to function effectively. In some embodiments such as the one presented, the top groove and the bottom groove may be integral, forming a continuous groove that interacts with both the top endplate 200 and the bottom endplate 300. The groove may be similar to a dovetail joint, providing a secure yet movable connection between the terminal body 100 and the endplates.

The terminal body 100 defines a terminal body aperture 118 that runs from the proximal surface of the terminal body 102 to the distal surface of the terminal body 104. In this embodiment, the terminal body aperture 118 is threaded, allowing for engagement with threaded members. In other embodiments, the terminal body aperture 118 may not be threaded but still configured to receive various types of securement members. The terminal body aperture 118 is configured to receive a threaded member, which may be a bolt, screw, or other threaded component.

The terminal body aperture 118 defines the central axis 16 of the spacer 10. The central axis 16 is used to reference points of expansion, serving as a baseline for the movement of other components during the expansion and contraction of the spacer 10.

The terminal body 100 provides structural support and alignment for the spacer 10. It facilitates the guided movement of the top endplate 200 and bottom endplate 300 through its grooves and interfaces with the driving member 400 and bolt 600 to enable expansion. The terminal body 100 may be constructed from biocompatible materials such as titanium alloys, stainless steel, or medical-grade polymers, ensuring strength and compatibility with the physiological environment.

The driving member 400 is located within the spacer 10, extending from the driving member proximal surface 402 to the driving member distal surface 404 along the central axis 16. It connects to both the bolt 600 and interfaces with the top endplate 200 and the bottom endplate 300. The driving member 400 includes a first side surface 406 and a second side surface 408, which extend between the proximal and distal surfaces. In the present embodiment, the first side surface 406 is opposite the central axis 16 than the second sider surface 408.

In the presented embodiment, the driving member 400 includes a plurality of guide rails. In some embodiments, a single guide rail may be sufficient to perform the necessary functions. Specifically, the present embodiment includes a first top guide rail 410, a second top guide rail 412, a third top guide rail 414, and a fourth top guide rail 416. It also includes a first bottom guide rail 418, a second bottom guide rail 420, a third bottom guide rail 422, and a fourth bottom guide rail 424. These guide rails are configured to be disposed within corresponding guide tracks. They are configured to move along the guide tracks when the driving member 400 experiences a force in the direction of the central axis 16. The guide rails may be protruding members and are configured to slide within the corresponding guide tracks. The guide rails may be slightly smaller than the guide tracks, allowing for some play between the guide track and the rail.

In the presented embodiment, the first top guide rail 410 is spaced apart from the second top guide rail 412. The first and second top guide rails are spaced apart axially with respect to the central axis 16. The first top guide rail 410 is on an opposite side surface than the third top guide rail 414 and the fourth top guide rail 416. The first and third top guide rails are on opposing sides of the central axis 16.

The driving member 400 includes each of the following ramped surfaces corresponding to the guide rails. The driving member first top ramped surface 426 corresponds to the first top guide rail 410. The driving member second top ramped surface 428 relates to the second top guide rail 412. The driving member third top ramped surface 430 is associated with the third top guide rail 414. The driving member fourth top ramped surface 432 corresponds to the fourth top guide rail 416. Similarly, the driving member first bottom ramped surface 434 corresponds to the first bottom guide rail 418. The driving member second bottom ramped surface 436 relates to the second bottom guide rail 420. The driving member third bottom ramped surface 438 is associated with the third bottom guide rail 422. The driving member fourth bottom ramped surface 440 corresponds to the fourth bottom guide rail 424. These ramped surfaces may allow for easier translation between the guide rails and the guide tracks.

Additionally, the driving member 400 includes each of the following faces. The driving member top first ascending ramped face 446 is one such face. The driving member top first descending ramped face 448 is another. The driving member top first neutral face 450 is also included. The driving member top second ascending ramped face 452 is part of the driving member 400. The driving member top second descending ramped face 454 and the driving member top second neutral face 456 are also included. The driving member bottom first ascending ramped face 458 is present on the driving member 400. The driving member bottom first descending ramped face 460 and the driving member bottom first neutral face 462 are additional faces. The driving member bottom second ascending ramped face 464, the driving member bottom second descending ramped face 466, and the driving member bottom second neutral face 468 are also part of the driving member 400.

The faces driving member top first ascending ramped face 446, driving member top first descending ramped face 448, driving member top first neutral face 450, driving member bottom first ascending ramped face 458, driving member bottom first descending ramped face 460, and driving member bottom first neutral face 462 are located at a proximal portion of the driving member 400. The faces driving member top second ascending ramped face 452, driving member top second descending ramped face 454, driving member top second neutral face 456, driving member bottom second ascending ramped face 464, driving member bottom second descending ramped face 466, and driving member bottom second neutral face 468 are located at a distal portion of the driving member 400.

The ascending ramped faces gain spatial distance from the central axis 16 when the driving member 400 is analyzed moving from the proximal end to the distal end. Conversely, the descending ramped faces lose spatial distance from the central axis 16 when the driving member 400 is analyzed moving from the proximal end to the distal end. These ramped faces may be configured to interact with the endplates, facilitating the expansion mechanism.

The angles of the ramps define ramp angles between the faces and the central axis 16. In the present embodiment, the ramp angles are the same. In other embodiments, the ramp angles of the driving member 400 may be different.

The driving member 400 includes both a first driving aperture 442 and a second driving aperture 444. The first driving aperture 442 is defined by a proximal portion of the driving member 400, while the second driving aperture 444 is defined by a distal portion of the driving member 400. In the present embodiment, neither the first nor the second driving apertures are threaded. In some embodiments, one or both of the first and second driving apertures may be threaded.

The driving member 400 is configured to translate rotational motion from the bolt 600 into linear movement along the central axis 16, enabling the expansion or contraction of the spacer 10. The driving member 400 may be constructed from biocompatible materials such as titanium alloys, stainless steel, or medical-grade polymers, ensuring durability and compatibility with body tissues.

The bolt 600 is positioned within the spacer 10, extending along the central axis 16. It connects the driving member 400 to the terminal body 100, facilitating the expansion mechanism of the spacer 10. The bolt 600 includes a bolt proximal end 602 and a bolt distal end 604. The bolt proximal end 602 is disposed on the opposite end of the bolt 600 from the bolt distal end 604.

The bolt 600 includes a bolt headed portion 606. The bolt headed portion 606 is disposed at the proximal end of the bolt 600. The bolt headed portion 606 is configured to abut the driving member 400. This configuration allows force in the direction of the central axis 16 to be placed on the driving member 400 when the bolt 600 is rotated.

The bolt headed portion 606 defines a bolt headed pattern 610. The bolt headed pattern 610 may be of conventional medical design so that the bolt 600 can be rotated by standard medical tools. In some embodiments, the bolt headed pattern 610 may be configured to be unique, providing compatibility with specific instruments.

The bolt 600 includes a bolt threaded portion 608. The bolt threaded portion 608 is disposed at the distal end of the bolt 600. The bolt threaded portion 608 is configured to interact with the terminal body aperture 118 of the terminal body 100, which is threaded in this embodiment. When the bolt 600 is disposed in both the driving member 400 and the terminal body 100, it acts to attach the driving member 400 to the terminal body 100.

The spacer 10 includes a washer 500. The washer 500 is configured to be disposed between the bolt 600 and the driving member 400. The washer 500 allows force to spread across the driving member 400 from the bolt 600 more evenly. In the present embodiment, the washer 500 is a ring with a cutaway section. In some embodiments, the washer 500 may be a complete ring. In other embodiments, the washer 500 may have various shapes to suit specific functional requirements.

The bolt 600 transmits rotational motion into linear movement of the driving member 400 along the central axis 16, enabling the expansion or contraction of the spacer 10. The bolt 600 may be constructed from biocompatible materials such as stainless steel, titanium alloys, or other medical-grade metals to ensure strength, durability, and compatibility with body tissues.

The top endplate 200 is connected to the terminal body 100 at the terminal body top surface 106 and interfaces with the superior vertebral body. It extends from the top endplate proximal end 202 to the top endplate distal end 204 along the central axis 16. The top endplate proximal end 202 is on the opposite end of the top endplate 200 from the top endplate distal end 204. The top endplate 200 includes a top endplate outer surface 206, which is spaced apart from the central axis 16. The top endplate outer surface 206 defines a top endplate plane 208. The top endplate plane 208 and the central axis 16 define a top endplate plane angle 210. In the present embodiment, the top endplate plane angle 210 is 0 degrees. In embodiments where the top endplate plane angle 210 is 0 degrees, the top endplate outer surface 206 is parallel to the central axis 16.

The top endplate outer surface 206 includes a top endplate first panel 212 and a top endplate second panel 214. The top endplate first panel 212 is disposed at the top endplate proximal end 202. The top endplate first panel 212 includes the top endplate first protrusion 236 and the top endplate second protrusion 238. The top endplate first protrusion 236 is on an opposite side of the top endplate second protrusion 238 with respect to the central axis 16. The combination of the top endplate first protrusion 236 and the top endplate second protrusion 238 forms a top endplate slot 240. The top endplate slot 240 is sized to receive a portion of the driving member 400.

The top endplate second panel 214 is disposed at the top endplate distal end 204. The top endplate second panel 214 defines the top endplate first tongue 216 and the top endplate second tongue 218. In some embodiments, the top endplate 200 may only include a single tongue.

The top endplate 200 includes a top endplate first guide track 220 and a top endplate second guide track 222. In some embodiments, the top endplate 200 may include a single guide track. In the present embodiment, the top endplate first guide track 220 is ramped. The top endplate second guide track 222 is also ramped. In the present embodiment, the top endplate first guide track 220 has the same ramp angle as the top endplate second guide track 222. This allows the top endplate outer surface 206 to keep the same angle in comparison to the central axis 16. In embodiments where the ramp angle of the top endplate first guide track 220 is different from the ramp angle of the top endplate second guide track 222, during actuation, the top endplate plane angle 210 will change.

The top endplate 200 includes a top endplate first ascending ramped face 224, a top endplate first descending ramped face 226, and a top endplate first neutral face 228. The top endplate first neutral face 228 is disposed between the top endplate first ascending ramped face 224 and the top endplate first descending ramped face 226. The top endplate 200 also includes a top endplate second ascending ramped face 230, a top endplate second descending ramped face 232, and a top endplate second neutral face 234. The top endplate second neutral face 234 is disposed between the top endplate second ascending ramped face 230 and the top endplate second descending ramped face 232.

The top endplate 200 provides a surface for load distribution and maintains contact with the adjacent superior vertebral body. It interacts with the driving member 400 and the terminal body 100 to facilitate the expansion mechanism of the spacer 10. The top endplate 200 may be constructed from biocompatible materials such as titanium alloys, stainless steel, or medical-grade polymers, ensuring strength and compatibility with the physiological environment.

The bottom endplate 300 is connected to the terminal body 100 at the terminal body bottom surface 108 and interfaces with the inferior vertebral body. It extends from the bottom endplate proximal end 302 to the bottom endplate distal end 304 along the central axis 16. The bottom endplate proximal end 302 is on the opposite end of the bottom endplate 300 from the bottom endplate distal end 304.

The bottom endplate 300 includes a bottom endplate outer surface 306, which is spaced apart from the central axis 16. The bottom endplate outer surface 306 defines a bottom endplate plane 308. The bottom endplate plane 308 and the central axis 16 define a bottom endplate plane angle 310. In the present embodiment, the bottom endplate plane angle 310 is 0 degrees. In embodiments where the bottom endplate plane angle 310 is 0 degrees, the bottom endplate outer surface 306 is parallel to the central axis 16.

The bottom endplate outer surface 306 includes a bottom endplate first panel 312 and a bottom endplate second panel 314. The bottom endplate first panel 312 is disposed at the bottom endplate proximal end 302. The bottom endplate first panel 312 includes the bottom endplate first protrusion 336 and the bottom endplate second protrusion 338. The bottom endplate first protrusion 336 is on an opposite side of the bottom endplate second protrusion 338 with respect to the central axis 16. The combination of the bottom endplate first protrusion 336 and the bottom endplate second protrusion 338 forms a bottom endplate slot 340. The bottom endplate slot 340 may be sized to receive a portion of the driving member 400.

The bottom endplate second panel 314 is disposed at the bottom endplate distal end 304. The bottom endplate second panel 314 defines the bottom endplate first tongue 316 and the bottom endplate second tongue 318. In some embodiments, the bottom endplate 300 may only include a single tongue.

The bottom endplate 300 includes a bottom endplate first guide track 320 and a bottom endplate second guide track 322. In some embodiments, the bottom endplate 300 may include a single guide track. In the present embodiment, the bottom endplate first guide track 320 is ramped. The bottom endplate second guide track 322 is also ramped. In the present embodiment, the bottom endplate first guide track 320 has the same ramp angle as the bottom endplate second guide track 322. This allows the bottom endplate outer surface 306 to keep the same angle in comparison to the central axis 16. In embodiments where the ramp angle of the bottom endplate first guide track 320 is different from the ramp angle of the bottom endplate second guide track 322, during actuation, the bottom endplate plane angle 310 will change.

The bottom endplate 300 includes a bottom endplate first ascending ramped face 324, a bottom endplate first descending ramped face 326, and a bottom endplate first neutral face 328. The bottom endplate first neutral face 328 is disposed between the bottom endplate first ascending ramped face 324 and the bottom endplate first descending ramped face 326. The bottom endplate 300 also includes a bottom endplate second ascending ramped face 330, a bottom endplate second descending ramped face 332, and a bottom endplate second neutral face 334. The bottom endplate second neutral face 334 is disposed between the bottom endplate second ascending ramped face 330 and the bottom endplate second descending ramped face 332.

The bottom endplate 300 provides a surface for load distribution and maintains contact with the adjacent inferior vertebral body. It interacts with the driving member 400 and the terminal body 100 to facilitate the expansion mechanism of the spacer 10. The bottom endplate 300 may be constructed from biocompatible materials such as titanium alloys, stainless steel, or medical-grade polymers, ensuring strength and compatibility with the physiological environment.

Those with ordinary skill in the art will appreciate that various modifications and alternatives for the described and illustrated examples can be developed in light of the overall teachings of the disclosure, and that the various elements and features of one example described and illustrated herein can be combined with various elements and features of another example without departing from the scope of the invention. Accordingly, the particular examples disclosed herein have been selected by the inventor simply to describe and illustrate examples of the invention and are not intended to limit the scope of the invention or its protection, which is to be given the full breadth of the appended claims and any and all equivalents thereof.

What is claimed is:

1. An expandable spacer comprising:
a terminal body defining a top groove and a threaded terminal aperture which further defines a central axis;
a driving member defining a driving aperture, a first top rail and a first bottom rail;
a bolt having both a headed portion and a threaded portion, the bolt being disposed within both the terminal body and the driving member;
a top endplate defining a top endplate outer surface spaced apart from the central axis and having both a top tongue disposed within the top groove and a first top guide track in which the first top rail is disposed within, the first top guide track defining a first top ramp angle with respect to the central axis; and
a bottom endplate having a first bottom guide track in which the first bottom rail is disposed within,
wherein when the bolt is rotated, the headed portion of the bolt drives the top endplate along the first top ramp angle, increasing the space between the top endplate outer surface and the central axis.

2. The spacer of claim 1, wherein the terminal body further defines a bottom groove,
wherein the bottom endplate further defines a bottom endplate outer surface spaced apart from the central axis and having a bottom tongue disposed within the bottom groove, and
wherein the first bottom guide track further defines a first bottom ramp angle with respect to the central axis such that when the bolt is rotated, the headed portion of the bolt drives the bottom endplate along the first bottom ramp angle, increasing the space between the bottom endplate outer surface and the central axis.

3. The spacer of claim 2, wherein the top groove is integral with the bottom groove.

4. The spacer of claim 2, wherein an absolute value of first top ramp angle and the bottom ramp angle is the same such that when the bolt is rotated the rate of spatial increase between the top endplate outer surface and the central axis is the same as the rate of spatial increase between the bottom endplate and the central axis.

5. The spacer of claim 2, wherein an absolute value of the first top ramp angle and the bottom ramp angle is not the same such that when the bolt is rotated the rate of spatial increase between the top endplate outer surface and the central axis is not the same as the rate of spatial increase between the bottom endplate and the central axis.

6. The spacer of claim 1, wherein the top endplate outer surface further defines an endplate plane,
wherein the endplate plane and the central axis define a plane angle,
wherein the driving member further defines a second top rail spaced apart axial from the first top rail with respect to the central axis,
wherein the top endplate further defines a second top guide track spaced apart axial from the first top guide track with respect to the central axis, in which the second top rail is disposed within the second top guide track, the second top guide track defining a second top ramp angle with respect to the central axis.

7. The spacer of claim 6, wherein an absolute value of the first top ramp angle and the second top ramp angle is the same such that when the bolt is rotated the plane angle remains the same.

8. The spacer of claim 6, wherein an absolute value of the first top ramp angle and the second top ramp angle is not the same such that when the bolt is rotated, the plane angle does not remain the same.

9. The spacer of claim 1, wherein the combination of the top endplate, the bottom endplate, and the driving member define a fluid channel generally perpendicular to the central axis.

10. The spacer of claim 1 further comprising a washer configured to be disposed between the headed portion and the driving member.
